(19) United States
(12) Patent Application Publication
(10) Pub. No.: US 2011/0243849 A1
(43) Pub. Date: Oct. 6, 2011

(54) HEME-BINDING PHOTOACTIVE POLYPEPTIDES AND METHODS OF USE THEREOF

(76) Inventors: Michael A. Marletta, Berkeley, CA (US); Michael B. Winter, Berkeley, CA (US); Emily J. McLaurin, Seattle, MA (US); Steven Y. Reece, Cambridge, MA (US); Charles Olea, JR., San Diego, CA (US); Daniel G. Nocera, Winchester, MA (US)

(21) Appl. No.: 13/077,686

(22) Filed: Mar. 31, 2011

Related U.S. Application Data
(60) Provisional application No. 61/320,558, filed on Apr. 2, 2010.

(51) Int. Cl. A61K 49/00; C07K 14/795; C07K 14/805; C07K 14/80; C12N 9/96; C12P 21/00; G01N 21/75; C12N 13/00; C07K 19/00; B82Y 15/00

(52) U.S. Cl. 424/9.1; 530/400; 530/385; 530/401; 530/391.1; 435/188; 435/69.1; 435/7.1; 435/173.1; 435/69.6; 977/774; 977/920

(57) ABSTRACT

The present disclosure provides photoactive polypeptides. A subject photoactive polypeptide is useful in a variety of applications, which are also provided.

### CROSS-REFERENCE

This application claims the benefit of U.S. Provisional Patent Application No. 61/320,558, filed Apr. 2, 2010, which application is incorporated herein by reference in its entirety.

### STATEMENT REGARDING FEDERALLY SPONSORED RESEARCH

This invention was made with government support under Grant Nos. R01 GM070671 and R01CA126642-02, awarded by the National Institutes of Health; and under Grant No. W911NF-06-1-0101, awarded by the Army Research Office. The government has certain rights in the invention.

### BACKGROUND

Polypeptides that natively bind heme are under-utilized scaffolds for photoactive porphyrin-based tools. Photoactive porphyrins have broad applications, including serving as sensors for oxygen, sensitizers for photodynamic therapy, and probes for imaging. Traditional methods for photoactive porphyrin incorporation into heme-binding polypeptides have limited their utility as biological tools. Harsh, denaturing conditions are typically required to remove native heme from polypeptides, dramatically decreasing the number of viable polypeptide constructs. There is a need in the art for photoactive polypeptides suitable for use under biological conditions.

### LITERATURE

Vanderkooi et al. (1990) Biochem. 29:5332; Khajehpour et al. (2003) Proteins 53:656; WO 2007/139791.

### SUMMARY OF THE INVENTION

The present disclosure provides a photoactive polypeptide. A subject photoactive polypeptide is useful in a variety of applications, which are also provided.

### BRIEF DESCRIPTION OF THE DRAWINGS

FIG. 1 depicts a chemical structure of ruthenium(II) CO mesoporphyrin IX (RuMP).

FIG. 2 depicts the stability of Ru Tt H-NOX in mouse plasma at 37° C.

FIG. 3 depicts the crystal structure of Tt H-NOX containing RuMP solved at 2.00 Å resolution.

FIG. 4 depicts an overall alignment showing heme pocket residues of Ru Tt H-NOX and Tt H-NOX bound to its native protoporphyrin IX heme as an iron(II) oxy complex (molecule B, PDB 1U55).

FIG. 5 depicts an overall alignment showing secondary structural elements of Ru Tt H-NOX and heme-bound Tt H-NOX (molecule B, PDB 1U55).

FIG. 6 depicts steady-state absorbance and emission spectra of Ru Tt H-NOX and Ru Mb in aqueous HEPES/NaCl buffer.

FIG. 7 depicts a Stern-Volmer plot of the excited state lifetime of Ru Tt H-NOX vs. [O₂] showing linear phosphorescence quenching by O₂ from 0 to 256 μM O₂ (R²=0.9957).

FIG. 8 depicts steady-state absorbance and emission spectra of Pd Tt H-NOX.

FIG. 9 depicts a Stern-Volmer plot of the excited state lifetime of Pd Tt H-NOX vs O₂ concentration.

FIG. 10 depicts the stability of Pd Tt H-NOX in mouse plasma at 37° C.

FIG. 11 depicts steady-state absorbance and emission spectra of a Pd Tt H-NOX/QD conjugate.

### DEFINITIONS

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “polypeptide” includes fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” includes polypeptides comprising one or more of a fatty acid moiety, a lipid moiety, a sugar moiety, and a carbohydrate moiety. The term “polypeptides” includes post-translationally modified polypeptides.

A “photoactive” (or “photoreactive”) polypeptide is a polypeptide that is capable of responding to light. This “response” can include emission (e.g., luminescence), such as phosphorescence or fluorescence.

The terms “subject,” “individual,” “host,” and “patient” are used interchangeably herein to a member or members of any mammalian or non-mammalian species. Subjects and patients thus include, without limitation, humans, non-human primates, canines, felines, ungulates (e.g., equine, bovine, swine (e.g., pig)), avians, rodents (e.g., rats, mice), and other subjects. Non-human animal models, particularly mammals, e.g. a non-human primate, a murine (e.g., a mouse, a rat), lagomorpha, etc. may be used for experimental investigations.

“Treating” or “treatment” of a condition or disease includes: (1) preventing at least one symptom of the condition, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient, in combination with another agent, or alone in one or more doses, to effect such treatment for the disease. The “therapeutically effective amount” will vary depending on the compound, the disease and its severity and the age, weight, etc., of the subject to be treated.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more than one such excipient, diluent, carrier, and adjuvant.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polypeptides. The term “biological sample” encompasses a clinical sample, and also includes cells in culture, cell supernatants, cell lysates, serum, plasma, biological fluid, and tissue samples.

Before the present invention is further described, it is to be understood that this invention

not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a heme-binding photoactive polypeptide” or a “photoactive polypeptide” includes a plurality of such polypeptides and reference to “the prosthetic group” includes reference to one or more prosthetic groups and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a photoactive polypeptide. A subject photoactive polypeptide is useful in a variety of applications, which are also provided.

### Photoactive Polypeptides

A subject heme-binding photoactive polypeptide (also referred to herein as a “subject photoactive polypeptide”) can comprise a polypeptide (e.g., a heme-binding polypeptide) that includes bound thereto a prosthetic group and, within the prosthetic group, a metal center. The metal center can include a bound metal. A subject heme-binding photoactive polypeptide (also referred to herein as a “subject photoactive polypeptide”) can comprise a polypeptide (e.g., a heme-binding polypeptide) that includes bound thereto a prosthetic group without a metal center. A subject photoactive polypeptide has altered photoactivity when oxygen is near the prosthetic group (e.g., within about 5 Å, within about 4 Å, within about 3 Å, or within about 2 Å of the prosthetic group). A subject photoactive polypeptide thus provides a means of detecting the presence and/or level of oxygen in a cell or other environment. A subject photoactive polypeptide also provides a means of sensitizing singlet oxygen upon irradiation in the presence of oxygen for delivery to a cell or other environment. In some embodiments, a subject heme-binding photoactive polypeptide is a heme-binding luminescent polypeptide. In some embodiments, binding of oxygen enhances photoactivity (e.g., emission); in other embodiments, binding of oxygen reduces (e.g., quenches) photoactivity (e.g., emission).

A prosthetic group with bound metal (such as ruthenium porphyrin), or without metal bound, is photoactive; however, such a molecule is also toxic to living cells. A subject photoactive polypeptide exhibits photoactive properties, and is non-toxic to living cells and multicellular organisms. Furthermore a subject photoactive polypeptide exhibits superior photophysical properties, compared to the metal/prosthetic group complex or metal-free prosthetic group not bound to a polypeptide.

The prosthetic group/metal center present in a subject photoactive polypeptide is generally a prosthetic group/metal center that is not normally found in nature. The prosthetic group/metal center present in a subject photoactive polypeptide is a prosthetic group other than heme.

A subject photoactive polypeptide can detect oxygen in a concentration range of from about 200 nM to about 300 μM, e.g., from about 200 nM to about 500 nM, from about 500 nM to about from about 1 μM to about 4 μM, from about 4 μM to about 10 μM, from about 10 μM to about 50 μM, from about 50 μM to about 100 μM, from about 100 μM to about 150 μM, or from about 150 μM to about 300 μM. A subject photoactive polypeptide exhibits altered photoactivity (e.g. quenching) in a linear manner in a concentration range of from about 200 nM to about 250 μM. A subject photoactive polypeptide can detect oxygen with a precision of about ±0.11 mmHg.

A subject photoactive polypeptide can be administered to an individual (e.g., a mammalian subject or other organism) without substantial toxic effect, even when administered systemically (e.g., via intravenous administration) at concentrations of from about 10 mg/ml to about 20 mg/ml, from about 20 mg/ml to about 30 mg/ml, from about 30 mg/ml to about 40 mg/ml, from about 40 mg/ml to about 50 mg/ml, or from about 50 mg/ml to about 100 mg/ml.

### Heme-Binding Polypeptides

As noted above, a subject photoactive polypeptide comprises a polypeptide that can bind a prosthetic group. Such polypeptides are referred to herein as “heme-binding polypeptides.” In some embodiments, the heme-binding polypeptide is a naturally-occurring polypeptide, i.e., a polypeptide that is normally found in nature. In other embodiments, the heme-binding polypeptide is a non-naturally-occurring polypeptide. In some embodiments, a non-naturally-occurring heme-binding polypeptide is chemically synthesized. In some embodiments, a non-naturally-occurring heme-binding polypeptide is produced by a recombinant method. In some embodiments, the heme-binding polypeptide is a heme-binding fragment (domain) of a full-length heme-binding polypeptide. Virtually any polypeptide that is capable of binding a heme group (a prosthetic group) is suitable for use. In some embodiments, a suitable heme-binding polypeptide comprises a globin fold. In some embodiments, a suitable heme-binding polypeptide comprises a heme pocket.

Suitable heme-binding polypeptides include, but are not limited to, a hemoglobin polypeptide; a myoglobin polypeptide; a cytochrome P450 polypeptide; a catalase polypeptide; an oxygenase polypeptide; a haloperoxidase polypeptide; a peroxidase polypeptide; a cytochrome oxidase polypeptide (e.g., cytochrome c); a ferritin; an H-NOX (Herne Nitric oxide/OXygen binding) domain or H-NOX domain-containing protein from any organism, including, e.g., the thermophilic bacterium Thermoanaerobacter tengcongensis (Tt H-NOX). H-NOX domain-containing proteins include soluble guanylate cyclases (sGCs) and insoluble guanylyl cyclases. Exemplary H-NOX polypeptides are described in WO 2007/139791; such H-NOX polypeptides are suitable for use in a subject photoactive polypeptide. “H-NOX” is also known as “sensor of nitric oxide (“SONO”) and “heme nitric oxide binding” (“HNOB”).

Suitable heme-binding polypeptides include a heme-binding polypeptide comprising a wild-type amino acid sequence (e.g., an amino acid sequence of a naturally-occurring heme-binding polypeptide); and a heme-binding polypeptide comprising an amino acid sequence that differs from a wild-type or other reference amino acid sequence by 1 amino acid (aa), 2 aa, 3 aa, 4 aa, from 5 aa to about 10 aa, from about 10 aa to about 15 aa, or more than 15 aa.

### Prosthetic Groups

A subject photoactive polypeptide includes a prosthetic group other than heme (where heme consists of porphyrin with an iron atom contained at the center of the porphyrin ring). Suitable prosthetic groups include are generally multicyclic ring structures, and include porphyrin, a reduced porphyrin (e.g., chlorin), a chlorophyll, a chlorophyll derivative (e.g., phyropheophorbide, chlorin e6, chlorin p6 and purpurin 18), a synthetic chlorin (e.g., a benzoporphyrin derivative and purpurin), a bacteriochlorin (e.g., bacteriochlorophyll derivative), a synthetic bacteriochlorin, a porphyrin isomer (e.g., porphycence, heteroatom-fused porphyrin and inverted porphyrin), an expanded porphyrin (e.g., texaphyrin), and a porphyrin analog (e.g., phthalocyanine and naphthalocyanine). Suitable prosthetic groups include those disclosed in U.S. Patent Publication No. 2009/0149525.

Suitable prosthetic groups include Photofrin® (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis (o-propionamido phenyl)porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, δ-aminolevulinic acid, benzonaphthoporphyrazines, naturally occurring porphyrins, ALA-induced protoporphyrin IX, synthetic dichlorins, bacteriochlorins of the tetra(hydroxyphenyl)porphyrin series, purpurins, tin and zinc derivatives of octaethylpurpurin, etiopurpurin, tin-etio-purpurin, porphycenes, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, chlorine e₆ monoethylendiamine monamide, verdins such as, but not limited to zinc methylpyroverdin (ZNMPV), copro II verdin trimethyl ester (CVTME) and deuteroverdin methyl ester (DVME), pheophorbide derivatives, pyropheophorbide compounds, and texaphyrins.

Porphyrins, hydroporphyrins, benzoporphyrins, and derivatives are all related in structure to hematoporphyrin, and are suitable for use. Chlorins and bacteriochlorins are also porphyrin derivatives, and are suitable for use; these have the property of hydrogenated exo-pyrrole double bonds on the porphyrin ring backbone, allowing for absorption at wavelengths greater than 650 nm. Chlorins are derived from chlorophyll, and modified chlorins such as meta-tetra hydroxyphenylchlorin (mTHPC) have functional groups to increase solubility. Bacteriochlorins are derived from photosynthetic bacteria and are further red-shifted to about 740 nm.

Purpurins, porphycenes, and verdins are also porphyrin derivatives; and are suitable for use. Purpurins contain the basic porphyrin macrocycle, but are red-shifted to about 715 mm. Porphycenes have similar absorption wavelengths as hematoporphyrin (about 635 nm), and are synthetic stable compounds with avidity for cancerous tumors. Verdins contain a cyclohexanone ring fused to one of the pyrroles of the porphyrin ring. Phorbides and pheophorbides are derived from chlorophylls and can also be used. Texaphyrins are also suitable for use. A feature of texaphyrins is the presence of five, instead of four, coordinating nitrogens within the pyrrole rings. This allows for coordination of larger metal cations, such as trivalent lanthanides.

### Metal Centers

Suitable metals include, but are not limited to, ruthenium (Ru), palladium (Pd), platinum (Pt), tin (Sn), aluminum (Al), gadolinium (Gd), iridium (Ir), osmium (Os), rhenium (Re), and rhodium (Rh). In some embodiments, the metal is a platinum group metal, e.g., the metal is one of Ru, Rh, Pd, Os, Ir, and Pt.

### Fusion Polypeptides

In some embodiments, the heme-binding polypeptide is a fusion polypeptide that includes a fusion partner (also referred to as a “heterologous polypeptide”). Suitable fusion partners include, but are not limited to, polypeptides that confer enhanced stability in vivo (e.g., enhanced serum half-life); polypeptides that provide ease of purification, e.g., (His)ₙ, e.g., 6H is, and the like; polypeptides that provide for secretion of the fusion protein from a cell; an affinity domain; polypeptides that provide an epitope tag, e.g., glutathione-S-transferase (GST), hemagglutinin (HA; e.g., CYPYDVPDYA) (SEQ ID NO:1), FLAG (e.g., DYKDDDDK) (SEQ ID NO:2), c-myc (e.g., CEQKLISEEDL) (SEQ ID NO:3), and the like; polypeptides that provide a detectable signal, e.g., an enzyme that generates a detectable product (e.g., β-galactosidase, luciferase), or a protein that is itself detectable, e.g., a fluorescent protein, etc.; a protein transduction domain; polypeptides that provides for multimerization, e.g., a multimerization domain such as an Fc portion of an immunoglobulin; polypeptides that provide for targeting; polypeptides that provide for crossing the blood-brain barrier; polypeptides that provide for enhanced uptake into a cancer cell; antibodies that bind to tumor-associated antigens; a targeting ligand; and the like.

Exemplary affinity domains include His5 (HHHHH) (SEQ ID NO:4), His X6 (HHHHHH) (SEQ ID NO:5), C-myc (EQKLISEEDL) (SEQ ID NO:6), Flag (DYKDDDDK) (SEQ ID NO:7), StrepTag (WSHPQFEK) (SEQ ID NO:8), hemagluttinin, e.g., HA Tag (YPYDVPDYA) (SEQ ID NO:9), GST, thioredoxin, cellulose binding domain, RYIRS (SEQ ID NO:10), Phe-His-His-Thr (SEQ ID NO:11), chitin binding domain, S-peptide, T7 peptide, SH2 domain, C-end RNA tag, WEAAAREACCRECCARA (SEQ ID NO:12), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

In some embodiments, the fusion protein comprises a proteolytic cleavage site disposed between the heme-binding polypeptide and the fusion partner. Proteolytic cleavage sites include, but are not limited to, an enterokinase cleavage site: (Asp)₄Lys (SEQ ID NO:13); a factor Xa cleavage site: Ile-Glu-Gly-Arg (SEQ ID NO:14); a thrombin cleavage site, e.g., Leu-Val-Pro-Arg-Gly-Ser (SEQ ID NO:15); a renin cleavage site, e.g., His-Pro-Phe-His-Leu-Val-11e-His (SEQ ID NO:16); a collagenase cleavage site, e.g., X-Gly-Pro (where X is any amino acid); a trypsin cleavage site, e.g., Arg-Lys; a viral protease cleavage site, such as a viral 2A or 3C protease cleavage site, including, but not limited to, a protease 2A cleavage site from a picornavirus (see, e.g., Sommergruber et al. (1994) Virol. 198:741-745), a Hepatitis A virus 3C cleavage site (see, e.g., Schultheiss et al. (1995) J. Virol. 69:1727-1733), human rhinovirus 2A protease cleavage site (see, e.g., Wang et al. (1997) Biochem. Biophys. Res. Comm. 235:562-566), and a picornavirus 3 protease cleavage site (see, e.g., Walker et al. (1994) Biotechnol. 12:601-605.

A protein transduction domain (PDT) facilitates traversing a lipid bilayer, micelle, cell membrane, organelle membrane, or vesicle membrane. Exemplary protein transduction domains include but are not limited to a minimal undecapeptide protein transduction domain (corresponding to residues 47-57 of HIV-1 TAT comprising YGRKKRRQRRR) (SEQ ID NO:17); a polyarginine sequence comprising a number of arginines sufficient to direct entry into a cell (e.g., 3, 4, 5, 6, 7, 8, 9, 10, or 10-50 arginines); a VP22 domain (Zender et al., Cancer Gene Ther. 2002 June; 9(6):489-96); an Drosophila Antennapedia protein transduction domain (Noguchi et al., Diabetes 2003; 52(7):1732-1737); a truncated human calcitonin peptide (Trehin et al. Pharm. Research, 21:1248-1256, 2004); polylysine (Wender et al., PNAS, Vol. 97:13003-13008); RRQRRTSKLMKR (SEQ ID NO:18); Transportan GWTLNSAGYLLGKINLKALAALAKKIL (SEQ ID NO:19); KALAWEAKLAKALAKALAKHLAKALAKALKCEA (SEQ ID NO:20); and RQIKIWFQNRRMKWKK (SEQ ID NO:21). Exemplary PTDs include but are not limited to, YGRKKRRQRRR (SEQ ID NO:22), RKKRRQRRR (SEQ ID NO:23); an arginine homopolymer of from 3 arginine residues to 50 arginine residues. Exemplary PTD domain amino acid sequences include, but are not limited to: YGRKKRRQRRR (SEQ ID NO:24); RKKRRQRR (SEQ ID NO:25); YARAAARQARA (SEQ ID NO:26); THRLPRRRRRR (SEQ ID NO:27); and GGRRARRRRRR (SEQ ID NO:28).

Suitable fluorescent proteins that can be fusion partners include, but are not limited to, a green fluorescent protein from Aequoria victoria or a mutant or derivative thereof e.g., as described in U.S. Pat. Nos. 6,066,476; 6,020,192; 5,985,577; 5,976,796; 5,968,750; 5,968,738; 5,958,713; 5,919,445; 5,874,304; e.g., Enhanced GFP, many such GFP which are available commercially, e.g., from Clontech, Inc.; a red fluorescent protein; a yellow fluorescent protein; any of a variety of fluorescent and colored proteins from Anthozoan species, as described in, e.g., Matz et al. (1999) Nature Biotechnol. 17:969-973; and the like.

Suitable polypeptide fusion partners that provide for crossing the blood-brain barrier include a polypeptide that binds to an endogenous blood brain barrier (BBB) receptor. Suitable polypeptides that bind to an endogenous BBB include antibodies, e.g., monoclonal antibodies, or antigen-binding fragments thereof, that specifically bind to an endogenous BBB receptor. Suitable endogenous BBB receptors include, but are not limited to, an insulin receptor, a transferrin receptor, a leptin receptor, a lipoprotein receptor, and an insulin-like growth factor receptor. See, e.g., U.S. Patent Publication No. 2009/0156498.

Suitable fusion partners that provide for enhanced uptake into cancer cell include, but are not limited to, an androgen receptor ligand; an estrogen receptor ligand; a vasoactive intestinal peptide receptor ligand; a gastrin releasing peptide receptor agonist; a gastrin releasing peptide receptor antagonist; a somatostatin receptor agonist; a somatostatin receptor antagonist; an adrenocorticotropin hormone (ACTH) polypeptide; a corticotropin releasing factor (CRF) polypeptide; and the like. Suitable fusion partners that provide for enhanced uptake into cancer cell also include antibodies (include antigen-binding antibody fragments) that bind to a tumor-associated antigen.

As used herein, the term “targeting ligand” encompasses any agent that selectively binds to a cell or tissue to be treated with a subject photoactive polypeptide. In one embodiment, targeting ligands selectively bind to tumor tissue or cells versus normal tissue or cells of the same type. The targeting ligands in general may be ligands for cell surface receptors that are over-expressed in tumor tissue. Cell surface receptors over-expressed in cancer tissue versus normal tissue include epidermal growth factor receptor (EGFR) (overexpressed in anaplastic thyroid cancer and breast and lung tumors), metastin receptor (overexpressed in papillary thyroid cancer), ErbB family receptor tyrosine kinases (overexpressed in a significant subset of breast cancers), human epidermal growth factor receptor-2 (Her2/neu) (overexpressed in breast cancers), tyrosine kinase-18-receptor (c-Kit) (overexpressed in sarcomatoid renal carcinomas), HGF receptor c-Met (overexpressed in esophageal adenocarcinoma), CXCR4 and CCR7 (overexpressed in breast cancer), endothelin-A receptor (overexpressed in prostate cancer), peroxisome proliferator activated receptor delta (PPAR-delta) (overexpressed in most colorectal cancer tumors), PDGFR A (overexpressed in ovarian carcinomas), BAG-1 (overexpressed in various lung cancers), soluble type II TGF beta receptor (overexpressed in pancreatic cancer) folate and integrin (e.g., αvβ3).

Suitable fusion partners include polypeptides that comprise an arginine-glycine aspartic acid (RGD) sequence.

### Modifications

A subject photoactive polypeptide can comprise one or more modifications, depending, at least in part, on the application to which the subject photoactive polypeptide is to be used.

### Amino Acid Modifications

In some embodiments, a subject photoactive polypeptide comprises one or more non-naturally occurring and/or non-naturally-encoded amino acids. In some embodiments, the non-naturally encoded amino acid comprises a carbonyl group, an acetyl group, an aminooxy group, a hydrazine group, a hydrazide group, a semicarbazide group, an azide group, or an alkyne group. See, e.g., U.S. Pat. No. 7,632,924 for suitable non-naturally occurring amino acids.

Inclusion of a non-naturally occurring amino acid can provide for linkage to a polymer, a second polypeptide, a scaffold, etc. For example, a subject antibody linked to a water-soluble polymer can be made by reacting a water-soluble polymer (e.g., PEG) that comprises a carbonyl group to a subject photoactive polypeptide that comprises a non-naturally encoded amino acid that comprises an aminooxy, hydrazine, hydrazide or semicarbazide group. As another example, a subject photoactive polypeptide linked to a water-soluble polymer can be made by reacting a subject photoactive polypeptide that comprises an alkyne-containing amino acid with a water-soluble polymer (e.g., PEG) that comprises an azide moiety; in some embodiments, the azide or alkyne group is linked to the PEG molecule through an amide linkage.

Inclusion of a non-naturally encoded amino acid in a subject photoactive polypeptide can modulate the responsiveness (e.g., emission properties) of the photoactive polypeptide.

A “non-naturally encoded amino acid” refers to an amino acid that is not one of the 20 common amino acids or pyrolysine or selenocysteine. Other terms that may be used synonymously with the term “non-naturally encoded amino acid” are “non-natural amino acid,” “unnatural amino acid,” “non-naturally-occurring amino acid,” and variously hyphenated and non-hyphenated versions thereof. The term “non-naturally encoded amino acid” also includes, but is not limited to, amino acids that occur by modification (e.g. post-translational modifications) of a naturally encoded amino acid (including but not limited to, the 20 common amino acids or pyrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

### Therapeutic Agent

In some embodiments, a subject photoactive polypeptide is linked (covalently or non-covalently) to a therapeutic agent, e.g., a cancer chemotherapeutic agent; insulin; glucose; a hormone; and the like.

### Detectable Labels

In some embodiments, a subject photoactive polypeptide comprises a detectable label, including, e.g., an emissive (fluorescent or phosphorescent) label; a donor or a quencher suitable for use in FRET; a label enzyme; a radioisotope; a member of a specific binding pair; a quantum dot; and the like.

By “emissive label” is meant any molecule that may be detected via its inherent emission properties, which include emission detectable upon excitation. Suitable emissive labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 2002 Molecular Probes Handbook, 9th Ed., by Richard P. Haugland.

Förster resonance energy transfer (FRET) is phenomenon known in the art wherein excitation of one emissive dye is transferred to another without emission of a photon. A FRET pair consists of a donor chromophore and an acceptor chromophore (where the acceptor chromophore may be a quencher molecule). The emission spectrum of the donor and the absorption spectrum of the acceptor must overlap, and the two molecules must be in close proximity. The distance between donor and acceptor at which 50% of donors are deactivated (transfer energy to the acceptor) is defined by the Förster radius, which is typically 10-100 angstroms. Changes in the emission spectrum comprising FRET pairs can be detected, indicating changes in the number of that are in close proximity (i.e., within 100 angstroms of each other). This will typically result from the binding or dissociation of two molecules, one of which is labeled with a FRET donor and the other of which is labeled with a FRET acceptor, wherein such binding brings the FRET pair in close proximity.

Binding of such molecules will result in an increased emission of the acceptor and/or quenching of the fluorescence 15 emission of the donor. FRET pairs (donor/acceptor) useful in the invention include, but are not limited to, EDANS/fluorescein, IAEDANS/fluorescein, fluorescein/tetramethylrhodamine, fluorescein/Cy 5, IEDANS/DABCYL, fluorescein/QSY-7, fluorescein/LC Red 640, fluorescein/Cy 5.5 and fluorescein/LC Red 705.

In another aspect of FRET, an emissive donor molecule and a nonemissive acceptor molecule (“quencher”) may be employed. In this application, emission of the donor will increase when quencher is displaced from close proximity to the donor and emission will decrease when the quencher is brought into close proximity to the donor. Useful quenchers include, but are not limited to, DABCYL, QSY 7 and QSY 33. Useful fluorescent donor/quencher pairs include, but are not limited to EDANS/DABCYL, Texas Red/DABCYL, BODIPY/DABCYL, Lucifer yellow/DABCYL, coumarin/DABCYL and fluorescein/QSY 7 dye.

By “label enzyme” is meant an enzyme which may be reacted in the presence of a label enzyme substrate which produces a detectable product. Suitable label enzymes also include optically detectable labels (e.g., in the case of horse radish peroxidase (HRP)). Suitable label enzymes include but are not limited to, HRP, alkaline phosphatase, luciferase, and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989).

By “radioisotope” is meant any radioactive molecule. Suitable radioisotopes for use in the invention include, but are not limited to ¹⁴C, ³H, ³²P, ³³P, ³⁵S, ¹²⁵I, and ¹³¹I. The use of radioisotopes as labels is well known in the art.

By “partner of a binding pair” or “member of a binding pair” is meant one of a first and a second moiety, wherein said first and said second moiety have a specific binding affinity for each other. Suitable binding pairs include, but are not limited to, antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin.

### Antibodies

In some embodiments, a subject photoactive polypeptide is linked (covalently or non-covalently) to an antibody. An antibody can be linked to a subject photoactive polypeptide by chemical means or by recombinant means. As noted above, in some embodiments, a suitable antibody is one that binds a cancer cell.

In some embodiments, the antibody is an antigen-binding fragment. Examples of binding fragments include (i) a Fab fragment (a monovalent fragment consisting of the VL, VH, CL and CH1 domains; (ii) a F(ab′)₂ fragment (a bivalent fragment comprising two Fab fragments linked by a disulfide bridge at the hinge region; (iii) a Fd fragment (consisting of the VH and CH1 domains); (iv) a Fv fragment (consisting of the VH and VL domains of a single arm of an antibody); (v) a dAb fragment (consisting of the VH domain); (vi) an isolated CDR; (vii) a single chain Fv (scFv) (consisting of the VH and VL domains of a single arm of an antibody joined by a synthetic linker using recombinant means such that the VH and VL domains pair to form a monovalent molecule); (viii) diabodies (consisting of two scFvs in which the VH and VL domains are joined such that they do not pair to form a monovalent molecule; the VH of each one of the scFv pairs with the VL domain of the other scFv to form a bivalent molecule); (ix) bi-specific antibodies (consisting of at least two antigen binding regions, each region binding a different epitope).

### Polymers

In some embodiments, a subject photoactive polypeptide is linked to a polymer other than an amino acid polymer. Suitable polymers include, e.g., biocompatible polymers, and water-soluble biocompatible polymers. Suitable polymers include synthetic polymers and naturally-occurring polymers. Suitable polymers include, e.g., substituted or unsubstituted straight or branched chain polyalkylene, polyalkenylene or polyoxyalkylene polymers or branched or unbranched polysaccharides, e.g. a homo- or hetero-polysaccharide. Suitable polymers include, e.g., ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Suitable synthetic polymers include unsubstituted and substituted straight or branched chain poly(ethyleneglycol), poly(propyleneglycol) poly(vinylalcohol), and derivatives thereof, e.g., substituted poly(ethyleneglycol) such as methoxypoly(ethyleneglycol), and derivatives thereof. Suitable naturally-occurring polymers include, e.g., albumin, amylose, dextran, glycogen, and derivatives thereof.

In some embodiments, a subject photoactive polypeptide is modified with a poly(ethylene glycol) (PEG) polymer. Methods and reagents suitable for PEGylation of a protein are well known in the art and may be found in, e.g., U.S. Pat. No. 5,849,860. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

The PEG conjugated to the subject photoactive polypeptide can be linear. The PEG conjugated to the subject protein may also be branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

Suitable polymers can have an average molecular weight in a range of from 500 Da to 50000 Da, e.g., from 5000 Da to 40000 Da, or from 25000 to 40000 Da. For example, in some embodiments, where a subject antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

### Radiopaque Labels

A subject photoactive polypeptide will in some embodiments comprise a “radiopaque” label, e.g. a label that can be easily visualized using for example x-rays. Radiopaque materials are well known to those of skill in the art. The most common radiopaque materials include iodide, bromide or barium salts. Other radiopaque materials are also known and include, but are not limited to organic bismuth derivatives (see, e.g., U.S. Pat. No. 5,939,045), radiopaque multiurethanes (see U.S. Pat. No. 5,346,981), organobismuth composites (see, e.g., U.S. Pat. No. 5,256,334), radiopaque barium multimer complexes (see, e.g., U.S. Pat. No. 4,866,132), and the like.

### Crosslinking

A subject photoactive polypeptide can be covalently linked to a second moiety (e.g., a lipid, a polypeptide other than a subject photoactive polypeptide, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio)propinoamido butane (DPDPB).

### Methods of Making a Subject Photoactive Polypeptide

A subject photoactive polypeptide can be made by substituting a heme group (e.g., a prosthetic group with a bound iron molecule) with a prosthetic group/metal center or metal-free prosthetic group, as described above. A subject photoactive polypeptide can be made recombinantly, e.g., by genetically modifying a host cell (e.g., a prokaryotic host cell such as Escherichia coli; a eukaryotic cell, such as a yeast cell (e.g., Saccharomyces cerevisiae; Pichia; etc.), an insect cell, a mammalian cell, etc.) with a nucleic acid comprising a nucleotide sequence encoding a heme-binding polypeptide. In some embodiments, the host cell comprises one or more genetic modifications such that an endogenous heme biosynthetic pathway is functionally disabled. In some embodiments, the host cell comprises one or more genetic modifications that render the cell membrane permeable to an exogenously-provided prosthetic group/metal center complex. Methods for generating a subject photoactive polypeptide using recombinant methods are described in Example 1, below, and in Woodward et al. Nat. Methods (2007) 4:43-45.

In some embodiments, site-directed mutagenesis is used to generate a subject photoactive polypeptide having altered photoactivity. Methods for carrying out site-directed mutagenesis are well known in the art; and any such method can be used to generate a subject photoactive polypeptide having altered photoactivity (e.g., emission properties).

### Compositions

The present disclosure provides compositions, including pharmaceutical compositions, comprising a subject photoactive polypeptide. A subject composition can comprise, in addition to a subject photoactive polypeptide, one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES),2-(N-Morpholino)ethanesulfonic acid (MES), 2-(N-Morpholino)ethanesulfonic acid sodium salt (MES),3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; and the like.

The present disclosure provides compositions comprising a subject photoactive polypeptide and a pharmaceutically acceptable excipient. Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

As used herein, the terms “pharmaceutically acceptable carrier” and “pharmaceutically acceptable excipient” are used interchangeably, and include any material, which when combined with a subject photoactive polypeptide does not substantially affect the biological activity of the conjugate, does not induce an immune response in a host, and does not have any substantial adverse physiological effect on the host. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

The pharmaceutical compositions may be formulated for a selected manner of administration, including for example, topical, oral, nasal, intravenous, intracranial, intraperitoneal, intratumoral, peritumoral, subcutaneous, or intramuscular administration. For parenteral administration, such as subcutaneous injection, the carrier can comprise water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for a subject pharmaceutical composition. Suitable biodegradable microspheres are disclosed, for example, in U.S. Pat. Nos. 4,897,268 and 5,075,109.

In some embodiments, a subject pharmaceutical composition is administered parenterally, e.g., intravenously. Thus, the invention provides compositions for parenteral administration which comprise a subject conjugate dissolved or suspended in an acceptable carrier, e.g., an aqueous carrier, e.g., water, buffered water, saline, phosphate-buffered saline, and the like. The compositions may contain pharmaceutically acceptable auxiliary substances as required to approximate physiological conditions, such as pH adjusting and buffering agents, tonicity adjusting agents, wetting agents, detergents and the like.

A subject composition can be sterilized by conventional sterilization techniques, or may be sterile filtered. The resulting aqueous solutions can be packaged for use as is, or lyophilized, the lyophilized preparation being combined with a sterile aqueous carrier prior to administration. The pH of the preparations can range from 3 and 11, e.g., from about pH 5 to about pH 9, or from about pH 7 to about pH 8.

### Utility

A subject photoactive polypeptide finds use in a variety of applications.

In some embodiments, a subject photoactive polypeptide is used to detect an oxygen level in a living cell. The method generally involves contacting a subject photoactive polypeptide with a cell; and detecting photoactivity (such as emission), where the level of photoactivity is affected by the oxygen concentration. The method can be carried out in vivo in a multicellular organism (an individual). The method can be carried out in vitro in a living cell. The method can be carried out ex vivo, e.g, can be carried out on a tissue or cell obtained from an individual.

### Cancer Diagnosis and Treatment

Effective tumor treatment strategies can be improved with characterization of the tumor microenvironment, including metabolic profiling of factors such as pH and oxygen. Tumor metabolism depends on the recruitment of blood vessels from the host to supply oxygen and nutrients, and to remove waste products. As tumors develop, the blood vessels become leaky and poorly interconnected, resulting in an acidic, hypoxic, heterogeneous environment. Successful drug delivery to a tumor requires functional vasculature; and many cancer therapies need appropriate oxygen concentration in order to be maximally effective. For example, radiation, photodynamic therapy (PDT), and certain chemotherapeutic agents (e.g., bleomycin; actinomycin) are less effective in hypoxic tumor environments. Hypoxia increases the efficacy of other chemotherapeutic agents (e.g., bioreductive drugs, alkylating agents, etc.). A detailed knowledge of the oxygen levels in and around a tumor can assist not only in the profiling of tumor growth and metabolism, but also in improving treatment strategies.

The present disclosure provides a method of detecting an oxygen level in and around a tumor, the method generally involving contacting a tumor with a subject photoactive polypeptide; and detecting photoactivity (e.g. emission) of the polypeptide. A change in photoactivity is related to the amount of oxygen in or around the tumor. In some embodiments, a decrease in the photoactivity (e.g., emission properties; e.g., luminescence) is proportional to the amount of oxygen in or around the tumor.

### Photodynamic Therapy

A subject photoactive polypeptide is useful in conjunction with photodynamic therapy (PDT).

The present disclosure provides a method of treating a disease in an individual, the method generally involving: (a) administering to a target tissue of the individual a subject photoactive polypeptide; and (b) irradiating the target tissue, thereby killing the target tissue and treating the disease.

PDT methods are well documented in the patent and scientific literature, for example in WO 96/28412 and 98/30242. When using a subject photoactive polypeptide in PDT, abnormalities and disorders which can be treated include any malignant, pre-malignant and non-malignant abnormalities or disorders responsive to photochemotherapy, e.g. tumors or other growths, skin disorders such as psoriasis or actinic keratoses and acne, skin abrasions, and other diseases or infections e.g. bacterial, viral or fungal infections, for example Herpes virus infections.

Following administration of a subject photoactive polypeptide (e.g., topically or systemically), the area treated (cells, tissue, etc.) is exposed to light to achieve the desired effect, e.g. a photochemotherapeutic effect. The light irradiation step to activate a subject photoactive polypeptide may be effected according to techniques and procedures well known in the art. Suitable light sources capable of providing the desired wavelength and light intensity are well known in the art. The time for which the body surface or cells are exposed to light in the methods of the present invention may vary. Generally, the length of time for the irradiation step is in the order of minutes to several hours, from about 1 minute to about 5 minutes, from about 5 minutes to about 15 minutes, from about 15 minutes to about 30 minutes, from about 30 minutes to about 1 hour, or more than 1 hour. Appropriate light doses can be selected by a person skilled in the art and will depend on the amount of the photoactive polypeptide accumulated in the target cells or tissues. The irradiation will in general be applied at a dose level of 40 to 200 Joules/cm², for example at 100 Joules/cm² at a fluence range of less than 200 mW/cm². Irradiation with wavelengths of light in the range 300 nm-800 nm, e.g., from about 300 nm to about 500 nm, from about 500 nm to about 700 nm, from about 600 nm to about 800 nm, or other suitable range of wavelengths, can be used in conjunction with a subject method.

### Imaging Methods

A subject photoactive polypeptide can be used in an imaging method, e.g., to monitor trafficking on the cellular or organismal level. In some embodiments, a subject photoactive polypeptide can be used in a FRET-based assay.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Ru-Porphyrin Protein Scaffolds for Sensing Oxygen

### Materials

The following chemicals were used as received: triruthenium dodecacarbonyl (Ru₃(CO)₁₂), D-(+)-glucose, triethanolamine hydrochloride (TEA), deoxyribonuclease I (DNase I), dibasic sodium phosphate (Na₂HPO₄), benzamidine hydrochloride, imidazole, Tris base, trifluoroacetic acid (TFA) from Sigma-Aldrich; mesoporphyrin IX dihydrochloride from Frontier Scientific; ampicillin, terrific broth (TB), isopropyl-β-D-thiogalactopyranoside (IPTG), and N-2-hydroxyethyl piperazine-N′-ethanesulfonic acid (HEPES) from Research Products International Corp.; glacial acetic acid and dimethylsulfoxide (DMSO) from EMD Chemicals, Inc.; sodium chloride (NaCl) and glycerol from Fisher Scientific; 4-(2-aminoethyl)-benzenesulfonylfluoride hydrochloride (“Pefabloc”) from Biosynth International, Inc.; Bio-Rad Protein Assay from Bio-Rad Laboratories, Inc.; mouse plasma (with sodium citrate as an anticoagulant) from Innovative Research, Inc.; and Dulbecco's phosphate-buffered saline (DPBS) from Cellgro. DNA primers for polymerase chain reaction (PCR) amplification and site-directed mutagenesis were synthesized by Elim Biopharmaceuticals, Inc. and Integrated DNA Technologies, respectively.

### Procedures

Preparation of RuMP. Synthesis and all subsequent manipulations of ruthenium(II) CO mesoporphyrin IX (RuMP) were carried out in low light. RuMP was prepared through modification of published methods. Paulson, D. R.; Addison, A. W.; Dolphin, D.; James, B. R. J. Biol. Chem. 1979, 254, 7002-7006. Ru₃(CO)₁₂ (180 mg, 0.282 mmol) and mesoporphyrin IX dihydrochloride (60 mg, 0.094 mmol) were refluxed, under N₂, in ˜30 mL glacial acetic acid for 24-36 h. Reaction progress was monitored by UV-vis spectroscopy. The reaction product was then cooled in an ice bath, precipitated through the dropwise addition of chilled ddH₂O, and collected on Whatman grade 42 filter paper (55 mm, Whatman International Ltd.), yielding a deep red-purple solid, which was formed quantitatively, as judged by UV-vis spectroscopy. The product was washed with several volumes of chilled ddH₂O and dried overnight under house vacuum. ESI-MS Calcd. (Found): [M-H]⁻ 693.2 (693.2). NMR assignments for RuMP have been reported previously.⁴ Paulson, D. R.; Addison, A. W.; Dolphin, D.; James, B. R. J. Biol. Chem. 1979, 254, 7002-7006.

Plasmids for protein expression. The gene for Tt H-NOX (residues 1-188 of TtTar4H from Thermoanaerobacter tengcongensis) was polymerase chain reaction (PCR) amplified from an expression plasmid for insertion into the pCW vector. The 5′ oligonucleotide (oligo), GGA ATT CCA TAT GAA GGG GAC AAT CGT CGG (SEQ ID NO:29), was used to introduce a NdeI restriction endonuclease site. The 3′ oligo, GCT CTA GAT CAG TGG TGG TGG TGG TGG TG (SEQ ID NO:30), was used to introduce a XbaI restriction endonuclease site. The PCR product was double digested with XbaI and NdeI and ligated into the pCW plasmid. A stop codon was subsequently introduced at the 3′ end of the Tt H-NOX gene using site-directed mutagenesis following the Quikchange protocol (Stratagene). The 5′ oligo was GTT TTT GAG TAT AAG AAA AAT TGA GAG CAC CAC CAC CAC CAC CAC (SEQ ID NO:31), and the 3′ oligo was GTG GTG GTG GTG GTG GTG CTC TCA ATT TTT CTT ATA CTC AAA AAC (SEQ ID NO:32). Sequencing of the final Tt H-NOX construct was carried out by Elim Biopharmaceuticals, Inc.

The mouse Mb construct in the pCW plasmid was used as described previously. Paulson, D. R.; Addison, A. W.; Dolphin, D.; James, B. R. J. Biol. Chem. 1979, 254, 7002-7006. The construct contained an N-terminal hexa-histidine tag to aid in purification.

Protein expression and incorporation of RuMP. A modified method for unnatural porphyrin incorporation was used to express Tt H-NOX and Mb containing RuMP. Woodward, J. J.; Martin, N. I.; Marletta, M. A. Nat. Methods 2007, 4, 43-45. Overnight cultures of RP523 E. coli cells were grown anaerobically in Hungate tubes (Bellco Glass, Inc.) as described, (Woodward, J. J.; Martin, N. I.; Marletta, M. A. Nat. Methods 2007, 4, 43-45) except antibiotic selection was carried out with 75 μg/mL ampicillin. TB media (9 L) containing ampicillin was sparged overnight in a 10 L fermentor vessel with house N₂ gas that had been passed through a 0.22 μm filter (Millipore). Sterile 0.2% D-(+)-glucose was added to the media immediately prior to inoculation with the overnight culture. The expression culture was grown at 37° C. to an OD₆₀₀ of 0.8-1.0 in the fermentor vessel while being continuously sparged with N₂. The media was then cooled to 25° C. and the fermentor was covered in aluminum foil before the addition of 3-6 μg/mL RuMP (from a ˜200× stock in DMSO). Expression was induced with 1 mM IPTG, and induction was allowed to occur for 18-22 h at room temperature while the culture was continuously sparged with N₂. E. coli pellets were harvested in the dark and stored at −80° C. following centrifugation.

Purification of Ru Tt H-NOX and Ru Mb. Purification of Tt H-NOX (Olea et al. ACS Chem. Biol. 2008, 3, 703-710) and Mb (Woodward et al. Nat. Methods 2007, 4, 43-45) were carried out as described previously but with some modifications. All manipulations of Ru Tt H-NOX and Ru Mb during the purification were performed in the dark and/or behind aluminum foil.

For Ru Tt H-NOX, cell pellets (from 9 L of E. coli expression) were slowly thawed using warm water and re-suspended in −100 mL of buffer A (50 mM TEA, pH 7.5, 50 mM NaCl), which also contained 1 mM Pefabloc and DNase I. The resuspended cells were lysed 3 times with an EmulsiFlex-05 homogenizer (Avestin, Inc.) at 4° C. between 50,000 and 150,000 psi. The lysate was then heat-denatured at 70° C. for 30 min using a water bath. All further manipulations were carried out at 4° C. The lysate underwent centrifugation with an Optima XL-100K ultracentrifuge (Beckman Coulter, Inc.) for 1 h at 42,000 rpm. The supernatant was passed at 2 mL/min over a Toyopearl SuperQ-650M anion exchange column (Tosoh Bioscience GmbH) that had been equilibrated with buffer A. The flow-through was concentrated to ˜5 mL in a Vivaspin 20 10,000 MWCO PES spin concentrator (Sartorius Stedim Biotech). The protein (˜2.5 mL per run) was exchanged into buffer B (50 mM HEPES, pH 6.2, 5% glycerol) by gravity using a ˜100 mL Sephadex G-25 column. The protein was then applied at 0.5 mL/min to a Toyopearl CM-650M cation exchange column (Tosoh Bioscience GmbH) that had been equilibrated with buffer B. The column was washed at 0.5 mL/min with buffer B until the absorbance at 280 nm was steady. The protein was eluted with a NaCl gradient from 0% to 40% buffer C (50 mM HEPES, pH 6.2, 500 mM NaCl, 5% glycerol) over 90 mL while 2 mL fractions were collected. Fractions 25-40 were pooled and concentrated to less than 1 mL using a 10,000 MWCO spin concentrator. Finally, the concentrated protein underwent size-exclusion chromatography with a HiLoad 26/60 Superdex 75 column (GE Healthcare) that had been equilibrated with buffer A. The protein was separated with an isocratic flow of buffer A at 0.4 mL/min while 4 mL fractions were collected. Fractions 52-58 were pooled and concentrated with a 10,000 MWCO spin concentrator. Protein was stored at −80° C.

The yield of Ru Tt H-NOX was ˜5 mg/L of E. coli expression. Purity was estimated to be >90% by Coomassie stain following SDS-PAGE. ESI-MS for intact Ru Tt H-NOX Calcd. (Found): 22,012.4 (22,012). Purity of RuMP in isolated Ru Tt H-NOX was found to be 99% following protein denaturation by reversed phase HPLC (reported as percent of total peak area at 393 nm).

For Ru Mb, all manipulations were carried out at 4° C. or on ice. Cell pellets (from 9 L of E. coli expression) were slowly thawed and re-suspended in ˜100 mL of buffer D (50 mM sodium phosphate, pH 8.0, 200 mM NaCl, 1 mM benzamidine hydrochloride), which also contained 1 mM Pefabloc and DNase I. The resuspended cells underwent homogenization and centrifugation as described above. The supernatant was applied at 0.5 mL/min to a Ni-NTA Superflow column (Qiagen) that had been equilibrated with buffer D. The column was washed at 0.5 mL/min with buffer D containing 25 mM imidazole until the absorbance at 280 nm was constant. The protein was then eluted at 0.5 mL/min with buffer D containing 250 mM imidazole. The eluate was concentrated to ˜3 mL using a Vivaspin 20 3,000 MWCO PES concentrator and loaded onto the size-exclusion column described above. The protein was separated with an isocratic flow of buffer E (100 mM HEPES, pH 7.5, 50 mM NaCl) at 0.4 mL/min and 4 mL fractions were collected. Fractions 53-57 were combined and diluted ˜10-fold into buffer F (20 mM Tris, pH 8.0). The protein was then flowed at 5 mL/min over a POROS HQ/20 (10×100) anion exchange column (Applied Biosystems) equilibrated with buffer F. The eluate was concentrated and stored at −80° C.

The yield of Ru Mb was 0.5-1.0 mg/L of E. coli expression. Purity was estimated to be >90% by Coomassie stain following SDS-PAGE. ESI-MS for intact Ru Mb Calcd. (Found): 17,892.5 (17,891). Purity of RuMP in isolated Ru Mb was found to be 96-99% (determined as above).

Plasma Stability of Ru Tt H-NOX. Stability of Ru Tt H-NOX in mouse plasma was assessed by a modification of established methods. Barnikol, W. K. R.; Burkhard, O.; Poetzschke, H.; Domack, U.; Dinkelmann, S.; Guth, S.; Fiedler, B.; Manz, B. Comp. Biochem. Physiol, A Mol. Integr. Physiol. 2002, 132, 185-191. Purified Ru Tt H-NOX (1.5 mg/mL) was exchanged into DPBS using a PD-10 desalting column (GE Healthcare). The protein was incubated in equal volume mouse plasma at 37° C. in the dark. Plasma control samples containing equal volume DPBS in place of Ru Tt H-NOX (for background absorbance correction) were prepared in parallel. Samples were centrifuged at various time points (0, 4, 10, and 24 h) at 2,800 rpm for 5 min in a Beckman GS-6R centrifuge maintained at 4° C. An absorption spectrum was acquired of the supernatant for each sample.

Crystallization of Ru Tt H-NOX. All crystallization experiments for Ru Tt H-NOX were carried out in low light. Protein was exchanged into buffer G (20 mM TEA, pH 7.5) using a PD-10 column and then concentrated to ˜50-60 mg/mL using a Vivaspin 500 5,000 MWCO PES spin concentrator. Crystals were grown using sitting drop vapor diffusion in which 1 μL of the protein was mixed with 1 μL of reservoir solution and equilibrated against a 700 μL reservoir of 24% (w/v) PEG 3350 and 0.1 to 0.2 M lithium acetate at 20° C. Crystals appeared within 24 h. Cryoprotection was achieved by titrating glycerol into the drop reservoir until a final concentration of 12.5% (v/v) was reached. The crystals were flash frozen in liquid N₂ for storage.

### Physical Measurements

All mass spectra were acquired by the QB3/Chemistry Mass Spectrometry Facility at the University of California, Berkeley. A mass spectrum of RuMP was obtained in negative ion mode with a quadrupole time-of-flight (Q-Tof) mass spectrometer equipped with an electrospray ionization (ESI) source (Q-Tof Premier, Waters). Protein mass spectra were acquired on an Agilent 1200 liquid chromatograph (LC) that was connected in-line with a LTQ Orbitrap XL mass spectrometer equipped with an ESI source (Thermo). The LC was equipped with a C8 guard (Poroshell 300SB-C8, 5 μm, 12.5×2.1 mm, Agilent) and analytical (75×0.5 mm) columns. Solvent A was 0.1% formic acid/99.9% water and solvent B was 0.1% formic acid/99.9% acetonitrile (v/v). Following sample injection, analyte trapping was performed for 5 min with 99.5% A at a flow rate of 90 μL/min. The elution program consisted of a linear gradient from 30% to 95% B over 24.5 mM, isocratic conditions at 95% B for 5 min, a linear gradient to 0.5% B over 0.5 min, and finally isocratic conditions at 0.5% B for 9.5 min, at a flow rate of 90 μL/min. The column and sample compartments were maintained at 35° C. and 10° C., respectively. Positive ion mass spectra were recorded over the range m/z=500 to 2000. Measured charge state distributions were deconvoluted using ProMass software (version 2.5 SR-1, Novatia).

HPLC for determining both RuMP stochiometry and purity in the isolated proteins was carried out on a System Gold chromatograph (Beckman Coulter, Inc.). The LC was equipped with a 126 NMP Solvent Module, a diode array 168 NM Detector, and a Vydac C4 column (5 μm, 4.6 mm×250 mm). The RuMP-containing proteins (20-100 μM) were prepared in buffer H (50 mM HEPES, 7.4, 50 mM NaCl) and manually injected in 20 μL volumes. Solvent A was 0.1% TFA/99.9% water and solvent B was 0.05% TFA/99.95% acetonitrile. The elution profile consisted of a linear gradient from 0% to 100% solvent B over 20 mM, isocratic conditions at 100% B for 5 min, a linear gradient to 0% B over 5 min, and finally isocratic conditions at 0% B for 10 min at a flow rate of 1.0 mL/min. The peak area at 393 nm for the major eluting species (16.4 min retention time, 96-99% of total peak area) was integrated using 32 Karat software (version 7.0, Beckman Coulter, Inc.).

UV-vis absorption spectra were acquired on a Cary 3E, 300, or 5000 spectrophotometer (Varian). Steady-state emission spectra were recorded on an automated Photon Technology International (PTI) QM 4 fluorimeter equipped with a 150-W Xe arc lamp and a Hamamatsu R928 photomultiplier tube. Sample excitation was carried out at 550 nm. Time-resolved emission measurements were made with pump light provided by the third harmonic (355 nm) of a Quanta-Ray Nd:YAG laser (Spectra-Physics) running at 10 Hz. The pump light was passed through a BBO crystal yielding a visible frequency that was tuned to 550 nm and employed to excite samples. All lifetime values were collected in triplicate. Anaerobic protein samples were obtained by exchanging the protein into anaerobic buffer H using a PD-10 column while inside a glove bag (Coy Laboratory Products Inc.) maintained at 4° C. Samples were brought into the glove bag following 2 series of the following cycles: vacuum (20-25 in Hg), Ar, vacuum, Ar, vacuum, 90:10 high purity Ar:H₂ gas mixture. All gasses were purchased from Airgas. The proteins were kept anaerobic in a septum-sealed quartz cuvette. Aeration was achieved through slow addition of air to the sample followed by equilibration through careful mixing. O₂ values were quantified using a fiber optic O₂ sensing system (Ocean Optics) equipped with a ruthenium-based probe (FOXY).

The molar absorptivity of Ru Tt H-NOX and Ru Mb was obtained with UV-vis spectroscopy following denaturation of the proteins in 10:3:17 (v/v) pyridine:1M NaOH:H₂O. RuMP was used as a standard (ε=195 mM⁻¹ cm⁻¹ at 396 nm in pyridine/NaOH solution) for determination of porphyrin concentration.³

Time-resolved emission data for Ru Tt H-NOX were analyzed according to the Stern-Volmer equation for O₂ quenching:

$$\frac{{τ}_{0}}{τ} = 1 + {k}_{q} {τ}_{0} [ {O}_{2} ]$$

where τ₀ is the excited state lifetime in the absence of O₂, τ is the excited state lifetime at a certain O₂ concentration, and k_{q} is the bimolecular quenching constant.

Relative quantum yields of samples, Φₛₐₘ, were calculated using [Ru^{II}(bpy)₃](PF₆)₂ in water as the reference according to:

$${Φ}_{sam} = {Φ}_{ref} ( \frac{{A}_{ref}}{{A}_{sam}} ) ( \frac{{I}_{sam}}{{I}_{ref}} ) {( \frac{{η}_{sam}}{{η}_{ref}} )}^{2}$$

where A is the measured absorbance, η is the refractive index of the solvent, I is the integrated emission intensity, and Φ_{ref} is the emission quantum yield of the reference. Lakowicz, J. R. Principles of Fluorescence Spectroscopy, 3^{rd} ed.; Springer: New York, 2006. Φ_{ref} was 0.053 for Ru(bpy)₃²⁺. Henderson, L. J., Jr.; Cherry, W. R. J. Photochem. 1985, 28, 143-151. All absorption and emission data were acquired at room temperature in buffer H.

X-ray diffraction data were collected for the Ru Tt H-NOX crystals using synchrotron radiation at Beamline 8.3.1 at the Advanced Light Source, Lawrence Berkeley National Laboratory (Berkeley, Calif.). Diffraction images were collected at 100 K with 1 s exposure times and 1° oscillation per frame. Data were processed using the HKL2000 suite (Otwinowski, Z.; Minor, W. Method Enzymol. 1997, 276, 307-326) and molecular replacement was performed using Phaser (McCoy, A. J.; Grosse-Kunstleve, R. W.; Storoni, L. C.; Read, R. J. Acta Cryst. D Biol. Cryst. 2005, 61, 458-464) with PDB 1U55 as the search model. Model building was carried out using Coot (Emsley, P.; Cowtan, K. Acta Crystallogr. D Biol. Cryst. 2004, 60, 2126-2132) and refined using Phenix (Adams, P. D.; Grosse-Kunstleve, R. W.; Hung, L. W.; Ioerger, T. R.; McCoy, A. J.; Moriarty, N. W.; Read, R. J.; Sacchettini, J. C.; Sauter, N. K.; Terwilliger, T. C. Acta Cryst. D Biol. Cryst. 2002, 58, 1948-1954).The porphyrin ligand was refined using ruthenium mesoporphyrin as the model. Emsley, P.; Cowtan, K. Acta Crystallogr. D Biol. Cryst. 2004, 60, 2126-2132. The final Ru Tt H-NOX structure was refined to a R_{work} of 20.4% (R_{free} of 22.5%) at 2.00 A resolution. Root mean square deviation (rmsd) was calculated using CNS (Brunger, A. T.; Adams, P. D.; Clore, G. M.; DeLano, W. L.; Gros, P.; Grosse-Kunstleve, R. W.; Jiang, J. S.; Kuszewski, J.; Nilges, M.; Pannu, N. S.; Read, R. J.; Rice, L. M.; Simonson, T.; Warren. G. L. Acta Crystallogr. D Biol. Cryst. 1998, 54, 905-921) with Ru Tt H-NOX and PDB 1U55 (molecule B).

### Results

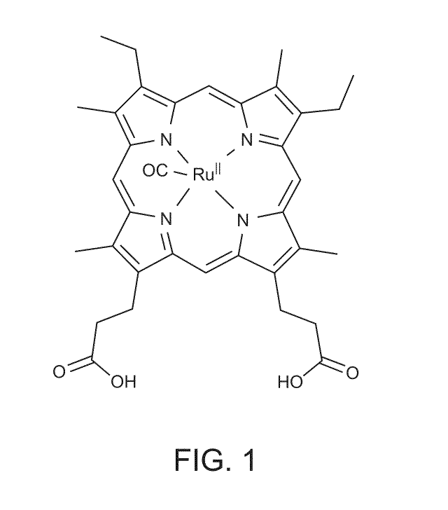

Ruthenium(II) CO mesoporphyrin IX (RuMP) (FIG. 1) is an ideal cofactor for protein-based sensors because it exhibits oxygen-sensitive phosphorescence and presents a proximal axial ligation site to facilitate binding to the protein scaffold. Myoglobin (Mb) and the H-NOX (Heme Nitric oxide/OXygen binding) domain from the thermophilic bacterium Thermoanaerobacter tengcongensis (Tt H-NOX) are robust proteins for RuMP sensors, as they can be readily modified with genetically-encoded affinity tags and site-directed mutagenesis. In addition, Tt H-NOX is stable under extreme temperatures (>70° C.).

FIG. 1. Chemical structure of ruthenium(II) CO mesoporphyrin IX (RuMP).

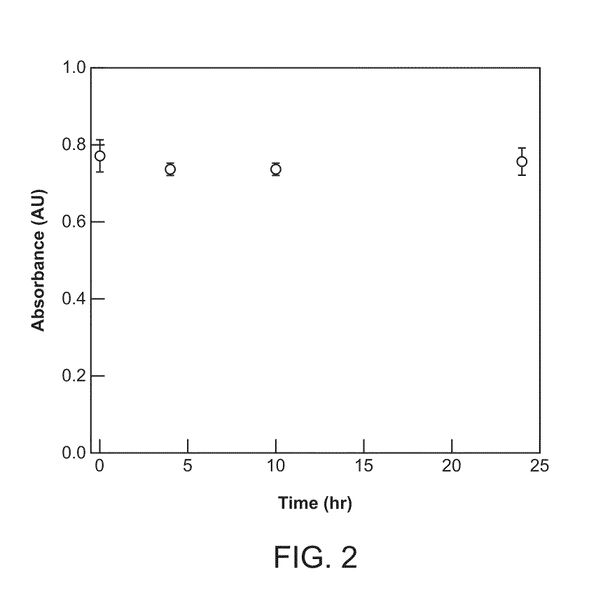

Experimental details for preparation and characterization of RuMP-substituted Mb (Ru Mb) and Tt H-NOX (Ru Tt H-NOX) are described in Materials and Methods. Briefly, RuMP was synthesized in a manner similar to published methods (Paulson, D. R.; Addison, A. W.; Dolphin, D.; James, B. R. J. Biol. Chem. 1979, 254, 7002-7006) and incorporated into Mb and Tt H-NOX during anaerobic protein expression. The RuMP-substituted proteins were isolated containing a stoichiometric amount of porphyrin (Table 1). Indeed, further evaluation of the stability of Ru Tt H-NOX indicated no detectible porphyrin loss for >24 hours under biological conditions (mouse plasma at 37° C., FIG. 2).

TABLE 1

| | HPLC | | UV-vis | |
|---|---|---|---|---|
| | A280 | Bradford | A280 | Bradford |
| Ru Tt H-NOX | 1.03 ± 0.04 | 1.17 ± 0.06 | 0.88 ± 0.01 | 1.13 ± 0.13 |
| Ru Mb | 0.67 ± 0.01 | 0.85 ± 0.05 | 0.61 ± 0.01 | 0.96 ± 0.12 |

Porphyrin content was quantified following denaturation of the proteins with reversed phase high performance liquid chromatography (HPLC) or in 10:3:17 (v/v) pyridine: 1M NaOH:H₂O (UV-vis assay).
Ru Mb (ε = 197 mM⁻¹ cm⁻¹ at 397 nm) and RuMP (ε = 195 mM⁻¹ cm⁻¹ at 396 nm) were used as porphyrin standards for the respective assays.
Protein concentration was determined using both the absorbance at 280 nm (ε = 30.9 mM⁻¹ cm⁻¹ for Tt H-NOX and 14.0 mM⁻¹ cm⁻¹ for Mb, ExPASy ProtParam tool) or the method of Bradford (Bradford, M. M. Anal. Biochem. 1976, 72, 248-254) (Bio-Rad Protein Assay).
All measurements were conducted in duplicate.

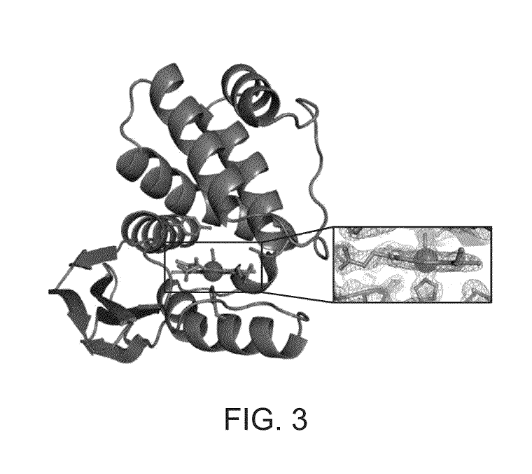

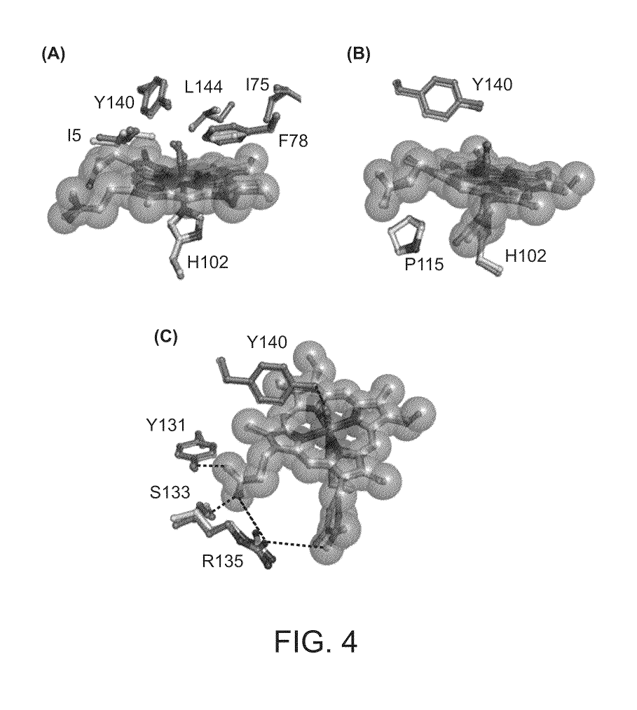

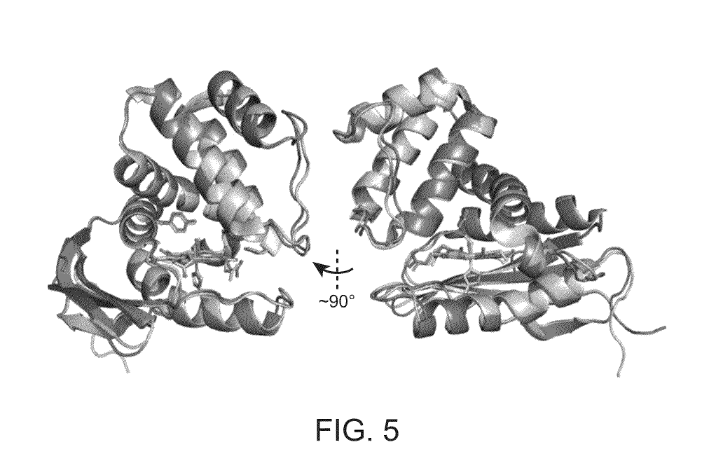

Purified Ru Tt H-NOX was crystallized to verify proper porphyrin insertion and preservation of the protein fold. The high-resolution (2.00 Å) structure of Ru Tt H-NOX (FIG. 3, Table 2) is the first crystal structure of a Ru porphyrin bound to a protein and demonstrates that the unnatural porphyrin maintains key contacts with surrounding heme pocket residues. These contacts include coordination of the proximal histidine to Ru and hydrogen bonding between the distal porphyrin ligand and a tyrosine residue (FIG. 3, FIG. 4). In fact, comparison of heme-bound Tt H-NOX with its Ru analogue indicates little perturbation of the protein fold (overall rmsd 1.3 Å, FIG. 5).

TABLE 2
Statistics of crystallographic data collection and refinement
for the Ru Tt H-NOX structure.
Data Collection

| Space group | P6₁22 |
|---|---|
| Cell dimensions | |
| a, b, c (Å) | 61.183, 61.183, 245.116 |
| α, β, γ(°) | 90, 90, 120 |
| Resolution (Å) | 50-2.00 (2.07-2.00) |
| R_{merge} (%)^{a} | 7.6 (30.7) |
| I/σ^{a} | 26.7 (10.1) |
| Completeness (%)^{a} | 99.0 (96.0) |
| Redundancy^{a} | 19.4 (19.4) |

Refinement

| | |
|---|---|
| No. of reflections | 19295 |
| R_{work}/R_{free}^{b} (%) | 20.4/22.5 |
| No. atoms | |
| Protein | 1504 |
| Porphyrin | 43 |
| CO molecules | 1 |
| Water molecules | 97 |
| B-factors | |
| Overall | 37 |
| Rms deviation | |
| Bond lengths (Å) | 0.013 |
| Bond angles (°) | 1.002 |

^{a}The values in parentheses relate to the highest resolution shells.
^{b}R_{free} is calculated for a randomly chosen 5% of reflections.

FIG. 2. Stability of Ru Tt H-NOX in mouse plasma at 37° C. Soret absorbance of Ru Tt H-NOX at 400 nm (corrected for plasma background) is plotted versus time. All samples were prepared in triplicate.

FIG. 3. Crystal structure of Tt H-NOX containing RuMP solved at 2.00 A resolution. Inset: 2(Fₒ-F_{c}) electron density map calculated by omitting RuMP and the proximal histidine side chain.

FIG. 4. Overall alignment showing heme pocket residues of Ru Tt H-NOX and

Tt H-NOX bound to its native protoporphyrin IX heme as an iron(II) oxy complex (molecule B, PDB 1U55). Pellicena, P.; Karow, D. S.; Boon, E. M.; Marletta, M. A.; Kuriyan, J. Proc. Natl. Acad. Sci. U.S.A. 2004, 101, 12854-12859. Van der Waals contacts are maintained between the unnatural porphyrin and I5, I75, F78, and L144 in the distal pocket (A) as well as with P115 in the proximal pocket (B). Hydrogen bonding interactions (dashed lines) are conserved between Y140 and the distal porphyrin ligand (CO or O₂) as well as between the porphyrin propionate groups and Y131, S133, and R135. Only the hydrogen bonding interactions for the Ru Tt H-NOX structure are shown for clarity (C).

FIG. 5. Overall alignment showing secondary structural elements of Ru Tt H-NOX and heme-bound Tt H-NOX (molecule B, PDB 1U55).

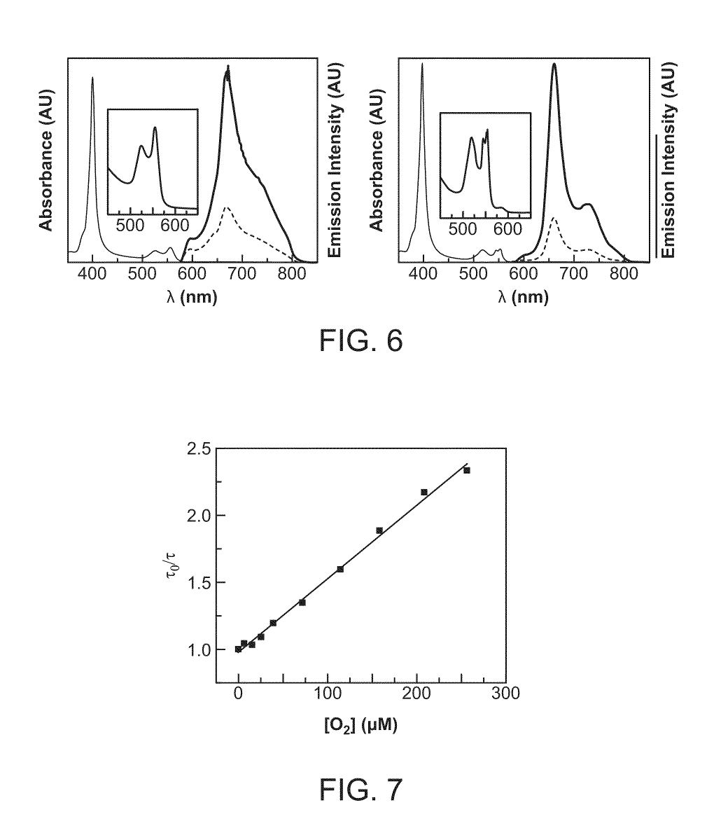

Steady-state and time-resolved spectroscopies were employed to examine the spectral properties of RuMP bound to the protein scaffolds. UV-visible spectra for Ru Tt H-NOX and Ru Mb show similar Soret band features at 400 nm and 397 nm, respectively (FIG. 6, Table 3). However, the α band at ˜550 nm for Ru Mb is split, as observed previously. Paulson et al. (1979) supra. Steady-state emission spectra reveal a blue-shifted emission band and decreased emission quantum yield for Ru Mb as compared to Ru Tt H-NOX (Table 3). Time-resolved emission spectroscopy conducted to further probe the spectral features of the porphyrin-protein complexes yielded single-exponential emission decays (following 550 nm laser excitation) under anaerobic conditions that vary widely between the two proteins (τₒ=7.7 μs for Ru Tt H-NOX vs. 37.3 μs for Ru Mb, respectively). Taken together, these data indicate a substantially different conformation and/or chemical environment for RuMP in Mb and Tt H-NOX. Indeed, the crystal structure of Mb reveals that the heme is partially exposed to solvent, whereas the heme in Tt H-NOX is buried within the protein matrix (FIG. 3).

TABLE 3

| Ru Protein | λ_{abs} (nm)/(ε) | λₑₘ^{a} (nm) | Φₑₘ^{c} | τₑₘ^{d} (μs) |
|---|---|---|---|---|
| Tt H-NOX | 400 (173) | 668 | 1.7 × 10⁻⁴ | 7.7 (−O₂) |
| | 524 (10.7) | ~734^{b} | | 2.9 (+O₂) |
| | 555 (13.9) | | | |
| Mb | 397 (197) | 663 | 4.8 × 10⁻⁵ | 37.3 (−O₂) |
| | 518 (12.2) | ~733^{b} | | 12.2 (+O₂) |
| | 553 (13.2) | | | |

^{a}λₑₓ = 550 nm.
^{b}shoulder.
^{c}λₑₓ = 550 nm, no O₂.
^{d}λₑₓ = 550 nm, λ_{det} = 640 nm, no O₂ and 256 μM O₂.

The ability of phosphorescent molecules to sense oxygen is determined by the degree of emission quenching in the presence of oxygen. Comparison of the steady-state emission spectra of Ru Tt H-NOX and Ru Mb measured under aerobic and anaerobic conditions reveals that oxygen appreciably quenches the emission of both proteins (FIG. 6). To further evaluate the highly stable Ru Tt H-NOX protein as an oxygen sensor, its excited state lifetime was measured at several oxygen concentrations (FIG. 7). The data were analyzed according to the Stern-Volmer (SV) equation for oxygen quenching and yielded a bimolecular quenching constant, k_{q}, of 1350 mmHg⁻¹ s⁻¹ (8.2×10⁸ M⁻¹ s⁻¹). In addition to intrinsic emission properties, the precision of lifetime-based oxygen sensors is governed by the instrument error associated with the lifetime measurement. Taking our instrument error of 2.5% into account, Ru Tt H-NOX can be used to determine oxygen concentrations to within ±2.5 mmHg (4.2 μM) in spite of its low quantum yield. This precision is comparable to that reported for commercial oxygen sensors (Table 4) and is ideally suited for determining oxygen concentrations in biology. Indeed, emission quenching was observed to be linear across the biologically relevant range of oxygen concentrations (FIG. 7).

TABLE 4

| | k_{q} | τ₀ | Precision^{a} | |
|---|---|---|---|---|
| Complex | (mmHg⁻¹s⁻¹) | (μs) | (mmHg) | Ref. |
| Ru Tt H-NOX | 1350 | 7.7 | 5.0 | this work |
| Ru^{II}(bpy)₃²⁺ | 4300 | 0.58 | 21 | 16 |
| Oxyphor | 293 | 707 | 0.25 | 17 |
| PtP-C343 | 150 | 60 | 5.9 | 12 |

^{a}Determined assuming an error of 2.5% in τ₀ measurement
Ref. 16: Oter, O.; Ribou, A. C. J. Fluoresc. 2009, 19, 389-397;
Ref. 17: Dunphy, I.; Vinogradov, S. A.; Wilson, D. F. Anal. Biochem. 2002, 310, 191-198;
Ref. 12: Finikova, O. S.; Lebedev, A. Y.; Aprelev, A.; Troxler, T.; Gao, F.; Garnacho, C.; Muro, S.; Hochstrasser, R. M.; Vinogradov, S. A. Chem. Phys. Chem. 2008, 9, 1673-1679.
^{a}Determined assuming an error of 2.5% in τ₀ measurement.

FIG. 6. Steady-state absorbance (thin line) and emission spectra of Ru Tt H-NOX (left) and Ru Mb (right) in aqueous HEPES/NaCl buffer. Emission spectra were acquired following excitation at 550 nm in the presence (dashed line, 256 μM) and absence (heavy line) of O₂.

FIG. 7. Stern-Volmer plot of the excited state lifetime of Ru Tt H-NOX vs. [O₂] showing linear phosphorescence quenching by O₂ from 0 to 256 μM O₂ (R²=0.9957).

The RuMP proteins described here represent a new class of sensors for detection of dissolved oxygen. The sensors are readily expressed in E. coli, exceptionally robust, and able to detect oxygen levels in the biologically relevant range. The photophysical properties may be further modulated with the choice of emissive porphyrin or through modification of the protein scaffold (e.g. via site-directed mutagenesis). In addition, the proteins may be expressed with genetically-encoded tags for targeted delivery in biology and derivatized for enhancing biocompatibility. This new class of sensors is useful for monitoring oxygen levels in biological contexts. One area of particular interest for sensing oxygen is in tumor microenvironments wherein detailed knowledge of local oxygen concentrations can improve cancer diagnosis and treatment.

### Example 2

### Pd-porphyrin Protein Scaffolds for O₂ Sensing

In order to generate protein scaffolds with enhanced emission properties, phosphorescent palladium(II) mesoporphyrin IX (Cowan, J. A.; Gray, H. B. Inorg. Chem. 1989, 28, 2074-8) was incorporated into the H-NOX domain from Thermoanaerobacter tengcongensis (Pd Tt H-NOX) during protein expression using the methodology described in Example 1. Purification of Pd Tt H-NOX was carried out with established protocols (Weinert, E. E.; Plate, L.; Whited, C. A.; Olea, C., Jr.; Marletta, M. A. Angew. Chem. Int. Ed. Engl. 2010, 49, 720-3) utilizing its C-terminal His₆ tag.

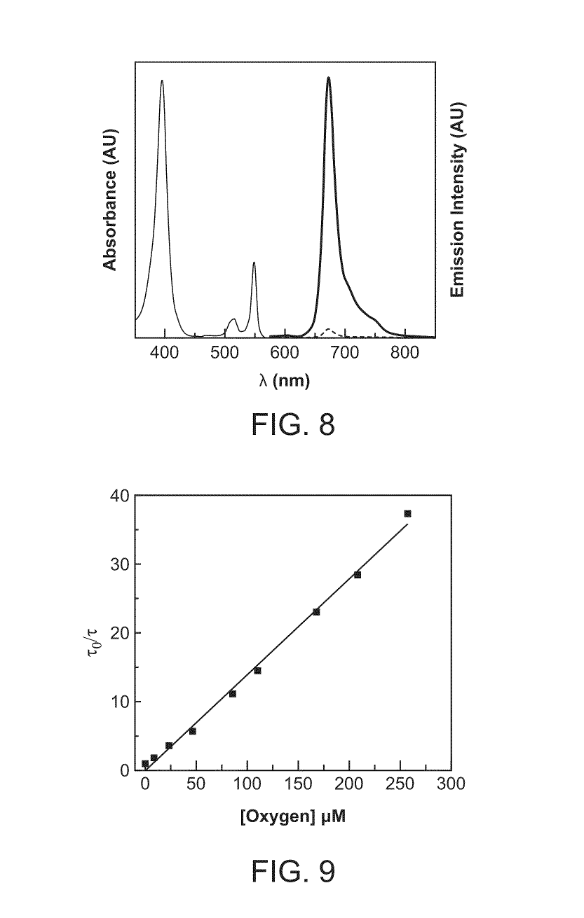

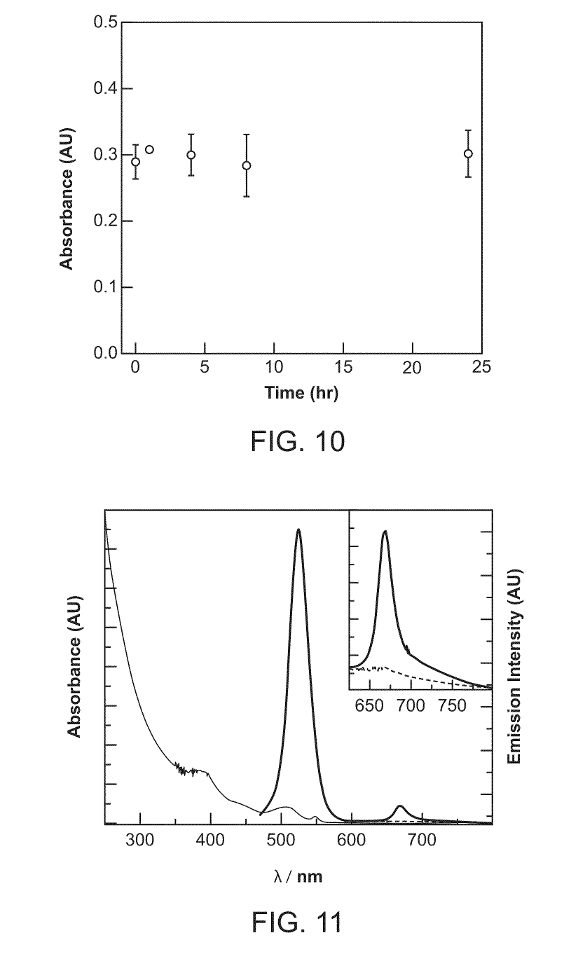

Purified Pd Tt H-NOX was characterized with steady-state absorption and emission spectroscopies. The Pd-containing protein displays a Soret band feature at 395 nm and bright emission at 672 nm in the absence of O₂ (FIG. 8 and Table 5). Comparison to Ru Tt H-NOX reveals an increased emission quantum yield (2.5×10⁻² vs. 1.7×10⁻⁴) and improved steady-state emission quenching in the presence of 256 μM O₂ (˜30 fold vs. ˜2.5 fold). Time-resolved emission spectroscopy indicates that Pd Tt H-NOX has a long-lived excited state (τ₀=483 μs) in the absence of O₂. A Stern-Volmer plot of the excited-state lifetime versus O₂ concentration was generated, revealing a linear O₂ response in the physiological range (FIG. 9). In addition, lifetime measurements indicate that Pd Tt H-NOX is capable of measuring O₂ levels with higher precision (±0.11 mmHg vs. ±2.5 mmHg O₂). Further characterization of Pd Tt H-NOX reveals that the construct is highly stable under biological conditions in mouse plasma (FIG. 10).

TABLE 5
Steady-state and time-resolved spectral properties of Pd Tt H-NOX

| | λ_{abs} | λₑₘ^{a} | | τ₀^{c} | k_{q} |
|---|---|---|---|---|---|
| Pd Protein | (nm) | (nm) | Φₑₘ^{b} | (μs) | (mmHg⁻¹s⁻¹) |
| Tt H-NOX | 395 | 672 | 2.5 × 10⁻⁴ | 483 | 465 |
| | 548 | | | | |
| | 515 | | | | |

^{a}λₑₓ = 550 nm.
^{b}λₑₓ = 550 nm, no O₂.
^{c}λₑₓ = 550 nm,
λ_{det} = 640 nm, no O₂

FIG. 8. Steady-state absorbance (thin line) and emission spectra of Pd Tt H-NOX. Emission spectra were acquired following excitation at 550 nm in the presence (dashed line, 256 μM) and absence (heavy line) of O₂.

FIG. 9. Stern-Volmer plot of the excited state lifetime of Pd Tt H-NOX vs. [O₂] showing linear phosphorescence quenching by O₂ from 0 to 256 μM O₂ (R²=0.9953).

FIG. 10. Stability of Pd Tt H-NOX in mouse plasma at 37° C. Soret absorbance of Pd Tt H-NOX at ˜395 nm (corrected for plasma background) is plotted versus time. All samples were prepared in triplicate. See Example 1 for experimental details.

### Example 3

### Coordination of a Pd-Porphyrin Protein Scaffold to a Quantum Dot

Pd Tt H-NOX was coordinated to a quantum dot (QD) to improve the photophysical properties of the Pd-containing protein for biological applications. QDs have numerous advantages for biological use, including high quantum yields, narrow emission line-widths, broad excitation profiles, and large two-photon absorption cross-sections. McLaurin, E. J.; Greytak, A. B.; Bawendi, M. G.; Nocera, D. G. J. Am. Chem. Soc. 2009, 131, 12994-13001.

Pd Tt H-NOX was coordinated via its C-terminal His₆ tag to dihydrolipoic acid (DHLA) QDs. Delehanty, J. B.; Medintz, I. L.; Pons, T.; Brunel, F. M.; Dawson, P. E.; Mattoussi, H. Bioconjug. Chem. 2006, 17, 920-7; Dif, A.; Boulmedais, F.; Pinot, M.; Roullier, V.; Baudy-Floc'h, M.; Coquelle, F. M.; Clarke, S.; Neveu, P.; Vignaux, F.; Le Borgne, R.; Dahan, M.; Gueroui, Z.; Marchi-Artzner, V. J. Am. Chem. Soc. 2009, 131, 14738-46. Excitation of the protein/QD conjugate at 350 nm resulted in excitation of the attached Pd Tt H-NOX protein, likely via Förster resonance energy transfer (FRET) from the QD (FIG. 11). Comparison of the steady-state emission spectra of the protein/QD conjugate in the absence and presence of O₂ (256 μM) reveals that Pd Tt H-NOX retains its O₂ sensitivity (FIG. 11, inset). Notably, the QD-based emission (−525 nm) is insensitive to O₂, indicating that the QD could serve as in internal standard for ratiometric O₂ detection. Excitation of the QD and subsequent energy transfer from the QD to the protein could serve as a strategy to enhance the brightness of the attached photoactive protein. In addition, two-photon excitation of the QD with lower energy light could improve biological functionality in deeper tissues.

FIG. 11. Steady-state absorbance (thin line) and emission spectra of the Pd Tt H-NOX/QD conjugate. Emission spectra were acquired following excitation at 350 nm in the presence (dashed line, 256 μM) and absence (heavy line) of O₂. The QD emission at ˜525 nm is not responsive to O₂, whereas the emission from Pd Tt H-NOX (−670 nm) is highly O₂-sensitive (inset).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A photoactive polypeptide comprising:
a) a heme-binding polypeptide; and
b) a prosthetic group, wherein the prosthetic group is bound to the heme-binding polypeptide, wherein the prosthetic group is not protoporphyrin or mesoporphyrin,
wherein the photoactivity of the photoactive polypeptide is altered upon exposure to oxygen.

2. The photoactive polypeptide of claim 1, wherein the prosthetic group comprises a metal center.

3. The photoactive polypeptide of claim 2, wherein the metal is ruthenium, palladium, platinum, tin, aluminum, gadolinium, iridium, osmium, rhenium, or rhodium.

4. The photoactive polypeptide of claim 1, wherein the photoactivity is emission.

5. The photoactive polypeptide of claim 4, wherein the photoactivity is quenched upon exposure to oxygen.

6. The photoactive polypeptide of claim 1, wherein the photoactivity is enhanced upon exposure to oxygen.

7. The photoactive polypeptide of claim 1, wherein the prosthetic group is a porphyrin, a reduced porphyrin, a chlorophyll, a chlorophyll derivative, a synthetic chlorin, a bacteriochlorin, a synthetic bacteriochlorin, a porphyrin isomer, an expanded porphyrin, a porphyrin analog, or a porphyrin derivative.

8. The photoactive polypeptide of claim 1, wherein the heme-binding polypeptide is a myoglobin, a hemoglobin, a cytochrome P450, a catalase, an oxygenase, a haloperoxidase; a peroxidase, a cytochrome, or an H-NOX-containing polypeptide.

9. The photoactive polypeptide of claim 8, wherein the heme-binding polypeptide is soluble guanylate cyclase.

10. The photoactive polypeptide of claim 1, wherein the heme-binding polypeptide is a non-naturally-occurring polypeptide.

11. The photoactive polypeptide of claim 1, wherein the photoactive polypeptide can detect oxygen to a concentration of about 200 nM.

12. The photoactive polypeptide of claim 1, wherein the photoactive polypeptide is a fusion polypeptide comprising a fusion partner.

13. The photoactive polypeptide of claim 12, wherein the fusion partner is an epitope tag, an antibody, an enzyme, a fluorescent protein, a protein transduction domain, an affinity domain, or a targeting polypeptide.

14. The photoactive polypeptide of claim 1, wherein the photoactive polypeptide comprises one or more modifications.

15. The photoactive polypeptide of claim 14, wherein the photoactive polypeptide comprises a detectable label.

16. The photoactive polypeptide of claim 15, wherein the detectable label is a dye, a radioisotope, a quantum dot, an enzyme, or a fluorescent protein.

17. The photoactive polypeptide of claim 14, wherein the photoactive polypeptide comprises a covalently or non-covalently linked non-polypeptide polymer.

18. The photoactive polypeptide of claim 17, wherein the polymer is poly(ethylene glycol).

19. The photoactive polypeptide of claim 14, wherein the photoactive polypeptide comprises a covalently or non-covalently linked therapeutic agent.

20. A method of producing a photoactive polypeptide of claim 1, the method comprising:
a) culturing a genetically modified host cell in a suitable medium, wherein the genetically modified host cell is genetically modified with a nucleic acid comprising a nucleotide sequence encoding the heme-binding polypeptide, wherein the genetically modified host cell is cultured under conditions such that the encoded heme-binding polypeptide is synthesized,
wherein the prosthetic group is bound to the encoded heme-binding polypeptide during the synthesis of the heme-binding polypeptide, and wherein the prosthetic group is not heme.

21. The method of claim 20, wherein the prosthetic group comprises a metal center.

22. The method of claim 21, wherein the metal is ruthenium, palladium, platinum, tin, aluminum, gadolinium, iridium, osmium, rhenium, or rhodium.

23. The method of claim 20, wherein the prosthetic group is a porphyrin, a reduced porphyrin, a chlorophyll, a chlorophyll derivative, a synthetic chlorin, a bacteriochlorin, a synthetic bacteriochlorin, a porphyrin isomer, an expanded porphyrin, a porphyrin analog, or a porphyrin derivative.

24. The method of claim 20, wherein the heme-binding polypeptide is a myoglobin, a hemoglobin, a cytochrome P450, catalase, an oxygenase, a haloperoxidase; a peroxidase, a cytochrome, or an H-NOX-containing polypeptide.

25. The method of claim 24, wherein the heme-binding polypeptide is a soluble guanylate cyclase.

26. A method of detecting an oxygen level in a cell or a tissue, the method comprising:
a) contacting the cell or tissue with a photoactive polypeptide of claim 1; and
b) detecting photoactivity, wherein the oxygen level alters the amount of photoactivity.

27. The method of claim 26, wherein said contacting is carried out in vivo.

28. The method of claim 26, wherein said contacting is carried out in vitro.

29. A method of sensitizing singlet oxygen in a cell or tissue, the method comprising:
a) contacting the cell or tissue with a photoactive polypeptide of claim 1; and
b) irradiating the photoactive polypeptide in the presence of oxygen.

30. The method of claim 29, wherein said irradiating results in killing of unwanted cells.

31. The method of claim 30, wherein the unwanted cells are cancer cells.

32. A method of imaging a cell, tissue, or other biological sample, the method comprising:
a) contacting the cell, tissue, or other biological sample with a photoactive polypeptide of claim 1; and
b) irradiating the photoactive polypeptide with light of a wavelength effective to induce photoactivity.

33. The method of claim 32, further comprising detecting the photoactivity.

34. The method of claim 32, wherein said contacting is carried out in vivo.


SEQUENCE LISTING (32 sequences)

SEQ ID NO: 1 - protein, 10 aa, Artificial Sequence
  Synthetic polypeptide
CYPYDVPDYA

SEQ ID NO: 2 - protein, 8 aa, Artificial Sequence
  Synthetic polypeptide
DYKDDDDK

SEQ ID NO: 3 - protein, 11 aa, Artificial Sequence
  Synthetic polypeptide
CEQKLISEEDL

SEQ ID NO: 4 - protein, 5 aa, Artificial Sequence
  Synthetic polypeptide
HHHHH

SEQ ID NO: 5 - protein, 6 aa, Artificial Sequence
  Synthetic polypeptide
HHHHHH

SEQ ID NO: 6 - protein, 10 aa, Artificial Sequence
  Synthetic polypeptide
EQKLISEEDL

SEQ ID NO: 7 - protein, 8 aa, Artificial Sequence
  Synthetic polypeptide
DYKDDDDK

SEQ ID NO: 8 - protein, 8 aa, Artificial Sequence
  Synthetic polypeptide
WSHPQFEK

SEQ ID NO: 9 - protein, 9 aa, Artificial Sequence
  Synthetic polypeptide
YPYDVPDYA

SEQ ID NO: 10 - protein, 5 aa, Artificial Sequence
  Synthetic polypeptide
RYIRS

SEQ ID NO: 11 - protein, 4 aa, Artificial Sequence
  Synthetic polypeptide
FHHT

SEQ ID NO: 12 - protein, 17 aa, Artificial Sequence
  Synthetic polypeptide
WEAAAREACCRECCARA

SEQ ID NO: 13 - protein, 5 aa, Artificial Sequence
  Synthetic polypeptide
DDDDK

SEQ ID NO: 14 - protein, 4 aa, Artificial Sequence
  Synthetic polypeptide
IEGR

SEQ ID NO: 15 - protein, 6 aa, Artificial Sequence
  Synthetic polypeptide
LVPRGS

SEQ ID NO: 16 - protein, 8 aa, Artificial Sequence
  Synthetic polypeptide
HPFHLVIH

SEQ ID NO: 17 - protein, 11 aa, Artificial Sequence
  Synthetic polypeptide
YGRKKRRQRRR

SEQ ID NO: 18 - protein, 12 aa, Artificial Sequence
  Synthetic polypeptide
RRQRRTSKLMKR

SEQ ID NO: 19 - protein, 27 aa, Artificial Sequence
  Synthetic polypeptide
GWTLNSAGYLLGKINLKALAALAKKIL

SEQ ID NO: 20 - protein, 33 aa, Artificial Sequence
  Synthetic polypeptide
KALAWEAKLAKALAKALAKHLAKALAKALKCEA

SEQ ID NO: 21 - protein, 16 aa, Artificial Sequence
  Synthetic polypeptide
RQIKIWFQNRRMKWKK

SEQ ID NO: 22 - protein, 11 aa, Artificial Sequence
  Synthetic polypeptide
YGRKKRRQRRR

SEQ ID NO: 23 - protein, 9 aa, Artificial Sequence
  Synthetic polypeptide
RKKRRQRRR

SEQ ID NO: 24 - protein, 11 aa, Artificial Sequence
  Synthetic polypeptide
YGRKKRRQRRR

SEQ ID NO: 25 - protein, 8 aa, Artificial Sequence
  Synthetic polypeptide
RKKRRQRR

SEQ ID NO: 26 - protein, 11 aa, Artificial Sequence
  Synthetic polypeptide
YARAAARQARA

SEQ ID NO: 27 - protein, 11 aa, Artificial Sequence
  Synthetic polypeptide
THRLPRRRRRR

SEQ ID NO: 28 - protein, 11 aa, Artificial Sequence
  Synthetic polypeptide
GGRRARRRRRR

SEQ ID NO: 29 - DNA, 30 bp, Artificial Sequence
  Synthetic oligonucleotide
ggaattccatatgaaggggacaatcgtcgg

SEQ ID NO: 30 - DNA, 29 bp, Artificial Sequence
  Synthetic oligonucleotide
gctctagatcagtggtggtggtggtggtg

SEQ ID NO: 31 - DNA, 45 bp, Artificial Sequence
  Synthetic oligonucleotide
gtttttgagtataagaaaaattgagagcaccaccaccaccaccac

SEQ ID NO: 32 - DNA, 45 bp, Artificial Sequence
  Synthetic oligonucleotide
gtggtggtggtggtggtgctctcaatttttcttatactcaaaaac